(19) United States
(12) Patent Application Publication
(10) Pub. No.: US 2005/0049716 A1
(43) Pub. Date: Mar. 3, 2005

(54) Bearing and composite structure

(76) Inventors: Sven Wagener, Essen (DE); Alfons Fischer, Essen (DE)

Correspondence Address: NIXON PEABODY, LLP, 401 9TH STREET, NW, SUITE 900, WASHINGTON, DC, 20004-2128, US

(21) Appl. No.: 10/496,573

(22) PCT Filed: Nov. 22, 2002
(86) PCT No.: PCT/EP02/13123

(30) Foreign Application Priority Data
Nov. 23, 2001 (DE) 101 57 316.2
Nov. 23, 2001 (DE) 101 57 315.4

(51) Int. Cl. A61F 2/30

(52) U.S. Cl. 623/23.5; 623/23.51

(57) ABSTRACT

A bearing, a composite structure, an implant and a method for producing a bearing with a micro-rough bearing surface are proposed. An improvement in the bearing properties—minimization of particle formation and reduction of friction—and a universally usable composite structure are achieved by the surface of the bearing or of the composite structure being roughened by etching and possibly provided with a coating, preferably consisting of plastic.

### BACKGROUND OF THE INVENTION

1. Field of the Invention

The present invention relates to a bearing, with a micro-rough bearing surface, a composite structure with a micro-rough surface, an implant with a bearing and a method for producing a bearing or a composite structure.

2. Description of Related Art

In the case of a bearing with a bearing surface, in particular a sliding bearing, a long service life with low friction is desired. Bearing surfaces made of metal, ceramic or plastic are frequently used.

With each of these, particle formation is especially problematical and, in the case of sliding bearings in particular, may lead to undesired three-body abrasion as it is known (bearing surface/particle/counterbearing surface). This may result in particular in an undesired formation of scratches, grooves or the like in the bearing surface and the counterbearing surface, with the consequence of a reduction in the service life. Furthermore, this may lead to undesired stiffness or an increase in friction.

In the case of an implant with a bearing surface, in particular a hip joint or the like, a long service life with low friction is desired. Bearing surfaces made of metal, ceramic or plastic are frequently used. The particle formation mentioned is particularly critical, since particles which become detached may lead to undesired side-effects in a body in which the implant is implanted.

### SUMMARY OF THE INVENTION

The present invention is based on the object of providing a bearing, a composite structure, an implant and a method for producing a bearing or composite structure such that the wear or abrasion can be minimized or at least can be reduced, that the formation of particles or at least the emergence of particles can be reduced and/or that the friction can be reduced, it being possible in particular for the service life of the bearing surface or other surface to be prolonged.

The above object is achieved by a bearing according to claim 1, a composite structure according to claim 12, an implant according to claim 17 or a method according to claim 18. Advantageous developments are the subject of the subclaims.

An underlying idea of the present invention is that of making the bearing surface have a micro-rough form, at least in a certain region or regions, preferably at least in the entire bearing or loading region, to be precise in a very simple and low-cost manner by etching.

“Micro-rough” is to be understood here as meaning that the surface is made to have a rough form—preferably into the μm range—in such a way that particles, preferably of up to 1 μm or even up to 10 μm, can be at least partly accepted by depressions in the surface, and in particular be embedded in them.

The micro-rough formation of the bearing surface leads to several advantages:

Firstly, particles occurring can be accepted in depressions, and in particular be permanently embedded in them. This applies in particular to very fine or micro-particles, which primarily occur when two surfaces slide on each other. In this way, three-body abrasion can be effectively reduced or even minimized.

Secondly, the micro-rough bearing surface can be adapted more easily to an assigned counterbearing surface. This is made possible in particular by plastic deformation or flattening of the micro-bumps. In this way, the bearing, preferably formed as a sliding bearing or joint, “runs in” more quickly.

Thirdly, the depressions of the micro-rough bearing surface can form a lubricant reservoir. This is conducive to reducing the friction and/or increasing the service life.

Fourthly, the micro-rough bearing surface is enlarged significantly in its surface area in comparison with a smooth surface. The enlarged surface area is better able to bind or retain particles and/or lubricant. This is in turn conducive to reducing the friction and/or prolonging the service life, in particular by reducing the three-body abrasion caused by free particles.

The micro-rough bearing surface is preferably made at least for the most part, in particular entirely, to have a macroscopically smooth form; consequently, the bearing surface appears to the human eye to be smooth, even if colorations or optical effects may possibly give the bearing surface an appearance of varying color.

In the case of an implant with a bearing of this type, the shedding of particles, and accordingly the potential adverse effects on a patient, can at least to a great extent be avoided or at least minimized by the acceptance and embedding of the particles that are produced especially during the initial running in of the bearing. The use of the bearing in the case of an implant or some other prosthesis therefore represents a particularly preferred use of the proposed bearing.

According to a particularly preferred design variant, the depressions of the micro-rough bearing surface are at least partially filled with a lubricant and/or plastic. This allows optimum, or at least significantly better, dry and/or emergency running properties of the bearing to be achieved. In particular, in this case significantly more favorable production is made possible than is the case for example with the known sintered bronzes with embedded lubricant.

According to a development of the design variant mentioned, the micro-rough bearing surface is provided with a preferably viscoelastic coating, which enters especially into the depressions and/or consists of plastic. On account of the micro-rough structure and the fine structure or nanostructure that preferably additionally forms as a result of the etching, an extremely durable composite comprising the micro-rough surface and the coating can be achieved. This composite can be used as a bearing surface or bearing part, the coating then preferably being formed from a material with at least adequate lubricating properties.

However, the composite structure mentioned can also be generally used for other workpieces, in particular heavy-duty workpieces. For example, the coating may have non-stick properties, so that the composite structure is suitable for example for producing cooking utensils or the like. Alternatively or additionally, the coating may for example have particularly corrosion-inhibiting or other preferred properties, so that correspondingly universal usability of the composite structure is obtained.

Further aims, advantages, properties and features of the present invention are explained in more detail below on the basis of the drawing, in which:

### BRIEF DESCRIPTION OF THE DRAWINGS

FIG. 1 shows a schematic representation of a proposed bearing with a micro-rough bearing surface, at least in a certain region or regions;

FIG. 2 shows a schematic sectional representation of an enlarged detail of the bearing surface, ignoring the curvature of the bearing surface existing in the case of the embodiment according to FIG. 1;

FIG. 3 shows an enlarged detail from FIG. 2;

FIG. 4 shows a proposed implant, formed as a hip joint; and

FIGS. 5a-5c show schematic sectional representations of the bearing surface or some other surface of various design variants.

### DETAILED DESCRIPTION OF THE INVENTION

In the figures, the same designations are used for identical or similar parts, corresponding or comparable advantages and properties being achieved even if the description is not repeated for reasons of simplification.

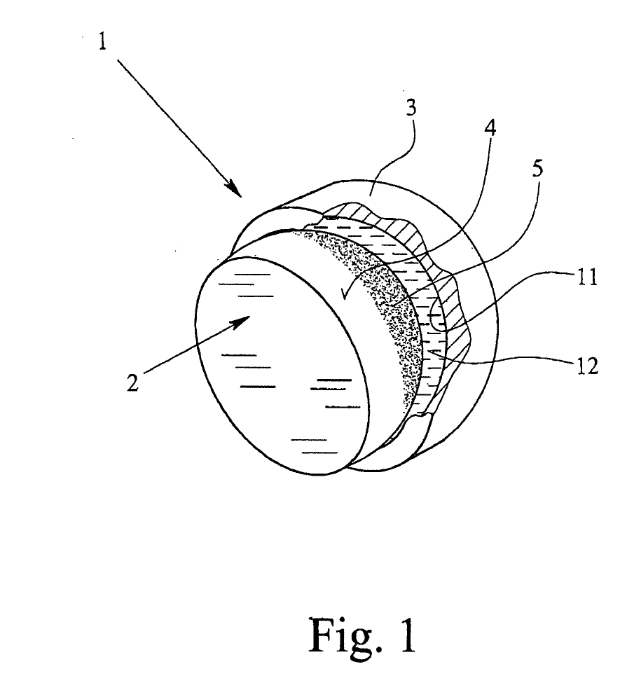

FIG. 1 shows, in a schematic representation a proposed bearing 1, which in the case of the example represented is formed as a sliding bearing. It may, however, also be some other bearing, such as a roller or rolling bearing.

The bearing 1 represented has a bearing head 2 and an assigned bearing shell 3, which for illustrative reasons are represented in FIG. 1 in the state in which they have been moved apart from each other. For purposes of illustration, the bearing shell 3 is also represented in section.

Instead of the formation as a bearing head 2 and bearing shell 3, the bearing elements assigned to each other may also have some other form, adapted to the respective intended use. In particular, a sliding and/or rolling mounting may be intended.

In the case of the example represented, the bearing 1 or its bearing head 2 has a preferably metallic bearing surface 4, which at least in one region 5, in particular at least in the entire rolling or bearing region, is made to have a micro-rough form. The micro-rough region 5 is represented in FIG. 1 as dotted for purposes of illustration.

In fact, the roughening of the bearing surface 4 in the region 5 or in the entire bearing surface 4 is formed so finely that the bearing surface 4 visually appears to be smooth to the human eye, even if the roughening gives the bearing surface 4 in the micro-rough region 5 the appearance of varying color.

The bearing 1 is produced from a suitable material or number of materials, such as metal, ceramic, plastic, composite material or the like.

The bearing surface 4 is preferably formed from a tough or ductile material. In particular, the bearing surface 4 is formed from plastic, ceramic or metal, preferably from steel, iron, titanium, chromium, an alloy based on iron, titanium or chromium and/or a cobalt-chromium alloy.

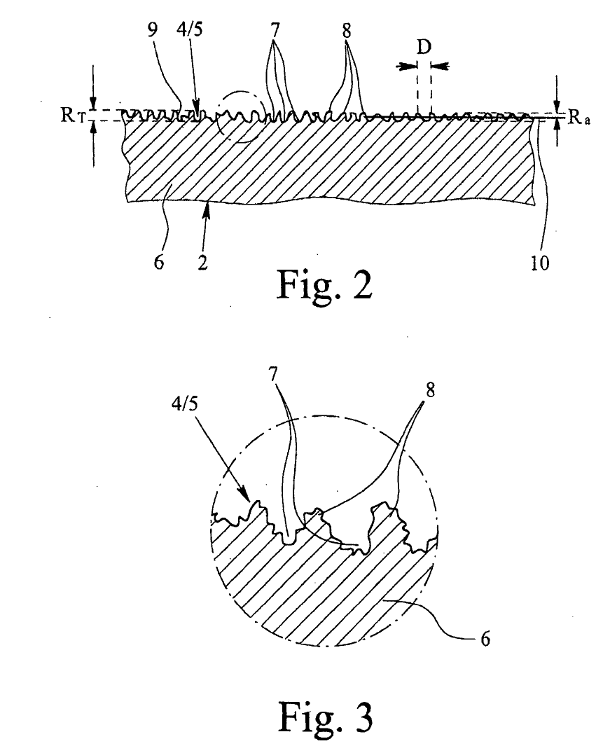

FIG. 2 shows in an enlarged sectional representation a detail of the bearing surface 4 with an adjoining surface layer 6 of the bearing head 2, the macroscopic curvature of the bearing surface 4, that is the spherical-head-like or dome-like formation of the bearing surface 4 having been omitted from the example represented to simplify the representation. Instead of this, in FIG. 2 the bearing surface 4 is represented as macroscopically planar.

For its nanostructuring, the micro-rough bearing surface 4 is provided with a multiplicity of depressions 7 and elevations 8, which merge or alternate with one another. In particular, the depressions 7 and elevations 8 merge with one another in such a way that there are at least substantially no planar surface portions formed in between.

The actual surface 9 of the micro-rough bearing surface 4 is accordingly significantly larger than the macroscopic area of extent of the bearing surface 4 provided by the macroscopically smooth contour 10. In the case of the example represented, the surface 9 is preferably at least 2×, in particular at least 2.5× or 3×, the macroscopic area of extent of the bearing surface 4.

The macroscopically smooth contour 10, only indicated on the right-hand side in FIG. 2, may be regarded as the intended profile, desired in the case of macroscopically customary machining, for example by cutting or grinding, which is preferably macroscopically smooth. In the case of the representation according to FIG. 2, for the purpose of explaining or defining the average roughness Rₐ, the contour 10 is not depicted on the elevations 8, but between the elevations 8 and the depressions 7. This is because the average roughness Rₐ represents the average deviation of the elevations 8 and depressions 7 from the average, macroscopically smooth intended surface or contour 10, as indicated in FIG. 2.

The two dashed lines on the right-hand side of FIG. 2 indicate the deviations in height of the depressions 7 and the elevations 8 from the average, macroscopically smooth contour 10. The determination of said average roughness Rₐ is based on these deviations.

Preferably, the average roughness Rₐ of the bearing surface 4 in the micro-rough region 5 is at least 1 nm, in particular at least 10 nm or 100 nm, and/or at most 10 μm, in particular up to 5 μm.

The peak-to-valley height R_{T}, i.e. the maximum difference in height between one of the elevations 8 and one of the depressions 7 in the micro-rough region 5, is preferably at most 20 μm, in particular a maximum of 10 μm.

The average diameter D of the depressions 7 is preferably at least 1 μm, in particular at least 2 or 5 μm and/or preferably at most 50 μm, in particular up to at most 20 or 10 μm. Most particularly preferred is a diameter D of 5 to 20 μm.

The elevations 8 and the depressions 7 are—depending on the application—more or less irregularly formed, as schematically indicated in FIG. 2. Preferably, the elevations 8 and depressions 7 are for their part again structured or made to have a rough form, in particular nanostructured, on their surface, as indicated by the schematic enlargement of a detail according to FIG. 3.

However, in principle, a regular or at least substantially uniform formation of the depressions 7 and/or the elevations 8 is also possible.

In spite of the irregularity mentioned, in the case of the micro-rough bearing surface 4 it is possible to speak of a profiling or structuring in the micrometer or nanometer range, i.e. in particular with structure widths of, for example, 100 nm to 50 μm. The structure width here designates the dimension by which individual structure elements, such as the depressions 7 or elevations 8, recur, i.e. for example the center-to-center distance of elevations 8 neighboring one another or depressions 7 neighboring one another.

The depressions 7 or elevations 8 are preferably arranged in an irregular distribution over the bearing surface 4, at least in the micro-rough region 5, the neighboring depressions 7 being separated from one another by preferably likewise irregularly formed elevations 8. In principle, however, at least substantially uniform distribution of the depressions 7 or the elevations 8 on the bearing surface 4 is also possible.

The average surface density of the depressions 7 or elevations 8 is preferably at least 1·10⁵/mm², in particular at least 2·10⁵/mm² or 5·10⁵/mm².

The bearing surface 4 is assigned a counterbearing surface 11, which in the case of the example represented is formed on the bearing shell 3, as indicated in FIG. 1. In the case of the example represented, the counterbearing surface 11 is formed such that it complements the bearing surface 4. However, the counterbearing surface 11 may—depending on the intended use and bearing structure—also have a form deviating from the complementary surface form. This applies in particular to other sliding bearings or roller or rolling bearings.

In the case of the example represented, the bearing surface 4 and the counterbearing surface 11 slide on each other, that is to say form a sliding bearing. However, rolling movements may also be superposed on the the sliding movement. As already mentioned above, other forms of bearing may in principle also be realized, for example with a planar bearing surface 4 and/or counterbearing surface 11 or with primarily rolling movement.

The counterbearing surface 11 is preferably made at least substantially to have a smooth form, that is to say preferably both macroscopically smooth and nanoscopically smooth (i.e. not micro-rough).

If need be, the counterbearing surface 11 may, however, also be made to have at least in a certain region or regions a micro-rough form. According to a design variant, the counterbearing surface 11 is provided with fine outwardly open pores or cavities, for example with an average diameter of 100 nm to 20 μm. In particular, the counterbearing surface 11 is in this case formed by an oxide film of a so-called valve metal (formation of the pores or cavities by anodizing), preferably aluminum oxide. The pores or cavities can then accept particles additionally occurring and/or serve as a lubricant reservoir.

The counterbearing surface 11 is formed from a suitable material, such as plastic, ceramic or metal. The counterbearing surface 11 is preferably harder than the bearing surface 4 or the micro-rough region 5 of the latter, in order to achieve the desired acceptance in the depressions 7 of the bearing surface 4 of particles occurring. In particular, the counterbearing surface 11 is formed from silicon dioxide or aluminium oxide. However, the counterbearing surface 11 may, for example, also be formed from the same or a similar material as the bearing surface 4.

In the case of the example represented, the bearing surface 4 that is micro-rough at least in a certain region or regions is formed on the bearing head 2 and the counterbearing surface 11 is formed on the bearing shell 3. However, this may also be reversed.

Depending on use, the bearing surface 4 and the counterbearing surface 11 may slide directly on each other, that is to say possibly form a lubricant-free mounting. Preferably, at least in the micro-rough region 5, the bearing surface 4 is assigned a lubricant 12, as indicated in FIG. 1.

The proposed bearing 1 is preferably used in such a way that the surface pressure of the bearing surface 4 or its region 5 is at most 100 MPa, in particular at most 50 MPa or 20 MPa. This applies in particular in the case of metallic formation of the bearing surface 4, but also depends on the material used.

The proposed micro-rough formation of the bearing surface 4, in particular in conjunction with a preferably at least substantially smooth and/or harder counterbearing surface 11, leads to the effect that very quick running-in is made possible, with low particle formation or at least low particle shedding. Moreover, relatively low friction is obtained. This can be explained by the fact that a rapid adaptation of the bearing surface 4, preferably formed from a tough and/or ductile material, in particular metal, to the counterbearing surface 11 takes place in the running-in phase, it being possible for particles occurring that may otherwise lead to undesired three-body abrasion to be accepted by the depressions 7 of the bearing surface 4. Moreover, the lubricant 12 adheres particularly well on the large surface area 9 of the bearing surface 4, a relatively large lubricant reservoir also forming in the depressions 7, so that low friction, in particular sliding friction, is made possible.

Tests have shown moreover that a further advantageous effect can occur in the case of the proposed solution. In particular in the case of metallic bearing surfaces 4, the metal particles occurring can—at least in a certain region or regions—form a very solid particle layer, of for example approximately 10 nm in thickness, on the elevations or micro-bumps 8. The particle layer forming can bond very well to the bearing surface 4 on account of the depressions 7. A high strength of the particle layer can be obtained in particular for the reason that, on account of their small size, the individual metal particles oxidize at least partially, in particular at least largely completely. A particularly hard layer, which is accordingly very wear-resistant or abrasion-resistant, then forms from the at least partially oxidized and/or ceramic-like particles.

The bearing surface 4 is roughened and/or structured by etching, in particular by sulfuric acid and/or chromosulfuric acid. This allows simple production. For example, the metallic bearing surface 4, consisting in particular of stainless iron or steel or a cobalt-chromium alloy, is exposed to the heated acid. When the acid is heated to approximately 200° C., for instance of one-molar concentration, an exposure time of 30 minutes to 2 hours is sufficient for example. Consequently, a wet-chemical treatment or roughening of the bearing surface 4 takes place.

There may additionally also be electrochemical support or promotion of the etching process. For example, warm acid at approximately 30° C. to 70° C., preferably approximately 40° C., is then sufficient with a comparable exposure time to etch the bearing surface 4 in a corresponding way, that is to remove it partially with the formation of the depressions 7.

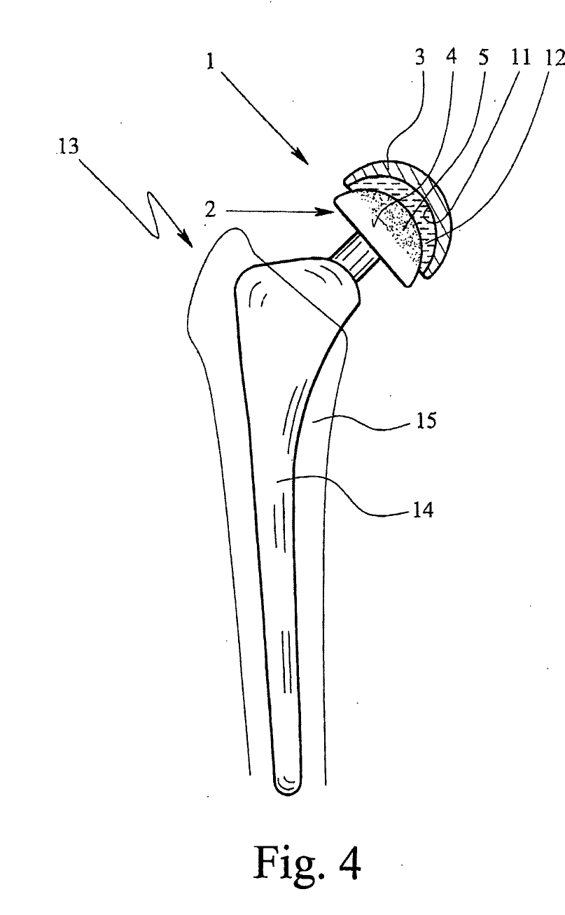

FIG. 4 shows in a schematic representation a proposed implant 13 which, in the case of the example represented, is formed as a joint, namely as a hip joint. However, it may for example also be some other joint, such as an artificial knee joint or some other implant performing a bearing function, or some other prosthesis with a joint.

The implant 13 represented forms an artificial hip joint. In the implantation, a stem 14 is inserted into a femur 15, indicated in FIG. 1, and the bearing shell 3 is inserted into an assigned region of the hip bone (not represented).

The proposed implant 13 or its bearing 1 exhibits the advantages already explained at length above. In particular, for the reasons mentioned, a service life that is significantly longer than in the case of conventional implants can be achieved.

Preferably, the implant 13 or its bearing 1 is used in such a way that the surface pressure of the bearing surface 4 or its region 5 is at most 100 MPa, in particular at most 50 MPa or 20 MPa.

Preferably, the bearing surface 4 is formed directly by the surface layer 6 or the carrier material of the bearing head 2 or some other bearing part. Consequently, an additional coating is preferably not provided to form the micro-rough bearing surface 4, and this makes correspondingly simple and low-cost production possible.

It is preferably provided that the etching for roughening or structuring the bearing surface 4 represents a final (shaping or mechanical) machining of the bearing surface 4. This is likewise conducive to simple and consequently low-cost production.

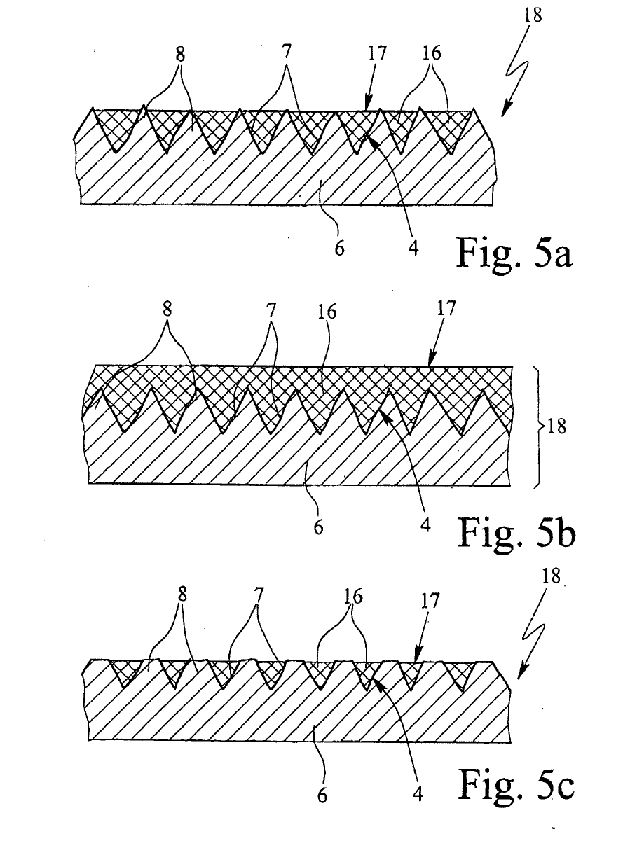

FIGS. 5a to c, show in schematic sectional representations the micro-rough bearing surface 4 with relatively uniform or regular structuring. In particular, the depressions 7 are formed at least substantially conically and/or the elevations 8 are formed at least substantially conically or frustoconically.

The depressions 7 are preferably filled at least partially with a material 16. In particular, this is a lubricant and/or a plastic. The material 16 is preferably made to be softer than the outer layer 6 or another material 16 forming the bearing surface 4.

The material 16 forms a coating 17, which at least partially covers over the bearing surface 4. In the case of the embodiment according to FIG. 5b, the coating 17 completely covers over the bearing surface 4.

In the case of the embodiment according to FIG. 5c, only partial coverage of the bearing surface 4 is provided in the case of frustoconically formed elevations 8. Here, the elevations 8 protect the material 16 located in the depressions 7 particularly well against mechanical effects.

The bearing surface forms with the material 16 or the coating 17 a proposed composite structure 18. An excellent composite with the material 16, in particular plastic, is made possible by the etching of the in particular metallic bearing surface 4. Consequently, the proposed composite structure 18 is extremely durable; the coating 17 consequently adheres extremely well on the bearing surface 4.

The proposed composite structure 18 is preferably intended for heavy-duty workpieces, in particular bearings or the like. However, the composite structure 18 may also be used for other purposes, in particular in the preferred combination of a metal surface 4 and plastic coating 17. Depending on the intended use, the surface 4 then does not represent a bearing surface, but some other surface of a workpiece.

For example, the material 16 or the coating 17 may be formed with a non-stick effect; the proposed composite structure 18 may then be used for example for cooking utensils or other articles preferably having non-stick properties.

Depending on the intended use, the material 16 is, in particular, PTFE (polytetrafluoroethylene), PFA (perfluoro-alkoxy polymers), PEEK (polyetherether ketone) or other suitable plastics, as cited in particular in the publication “Friction and wear of highly stressed thermoplastics bearings under dry sliding conditions” by S. Marx, R. Junghaus in Wear 193 (1996) 253-260, the entire content of which is hereby additionally incorporated in full as disclosure.

As already indicated, given appropriate selection, the material 16 may also act in particular as a lubricant, in order to achieve specific dry running and/or emergency running properties during use of the proposed composite structure 18 in the bearing 1. This is desirable particularly in the case of normally hydraulically lubricated bearings, in particular in mechanical engineering and automotive engineering, so that corresponding areas of use are obtained for the proposed bearing 1 or the proposed composite structure 18.

1-14. Cancel.

15. An implant, in particular a joint, such as a hip joint or knee joint, with a bearing, in particular a sliding bearing, with a bearing surface made micro-rough by depressions, the depressions being formed by etching of the bearing surface.

16. The implant as claimed in claim 15, wherein the depressions are arranged in an irregular distribution.

17. The implant as claimed in claim 15, wherein the bearing surface is made to have a macroscopically smooth form.

18. The implant as claimed in claim 15, wherein the bearing surface is finally (shapingly or mechanically) machined by the etching.

19. The implant as claimed in claim 15, characterized in that the bearing surface is formed from a tough material and/or from plastic, ceramic or metal, in particular from steel, iron, titanium, chromium, an alloy based on iron, titanium or chromium and/or a cobalt-chromium alloy.

20. The implant as claimed in claim 15, wherein the bearing surface has an averaged roughness or a peak-to-valley height of at most 20 μm, in particular up to 10 μm or 5 μm.

21. The implant as claimed in claim 15, wherein the bearing surface has at least substantially no planar surface portions between the depressions.

22. The implant as claimed in claim 15, wherein the depressions have an average diameter of from 1 to 50 μm, preferably from 5 to 20 μm.

23. The implant as claimed in claim 15, wherein the bearing surface is provided with the depressions with a surface density of at least 1·10⁵/mm².

24. The implant as claimed in claim 23, wherein the surface density is at least 2·10⁵/mm² or 5·10⁵/mm².

25. The implant as claimed in claim 15, wherein the bearing surface is assigned a counterbearing surface, which is harder than the bearing surface.

26. The implant as claimed in claim 15, wherein the depressions are formed at least substantially conically.

27. The implant as claimed in claim 15, wherein elevations between the depressions are formed at least substantially conically or frustoconically.

28. The implant as claimed in claim 15, wherein the depressions are at least partially filled with a lubricant, in particular plastic, preferably PTFE, PFA or PEEK.

29. The implant as claimed in claim 15, wherein the bearing surface is provided with a preferably viscoelastic coating, in particular made of plastic, preferably PTFE, PFA or PEEK.

30. A method for producing an implant with a micro-rough bearing surface, wherein the surface is roughened wet-chemically by etching and depressions in an irregular distribution are formed by the etching.

31. The method as claimed in claim 30, wherein the depressions are formed at least substantially conically.

32. The method as claimed in claim 30, wherein elevations between the depressions are formed at least substantially conically or frustoconically.

33. The method as claimed in claim 30, wherein the surface is formed from metal, ceramic or plastic, in particular from steel, iron, titanium, chromium, an alloy based on iron, titanium or chromium and/or a cobalt-chromium alloy.

34. The method as claimed in claim 30, wherein the surface is electrochemically etched.

35. The method as claimed in claim 30, wherein the etching is performed by exposure to acid, in particular heated acid, preferably chromosulfuric acid and/or sulfuric acid.

36. The method as claimed in claim 30, wherein the etching represents a final (shaping or mechanical) machining of the surface.
